(19) United States
(12) Patent Application Publication
(10) Pub. No.: US 2024/0366601 A1
(43) Pub. Date: Nov. 7, 2024

(54) METHODS FOR TREATING TRAUMATIC BRAIN INJURY

(71) Applicant: The Regents of the University of California, Oakland, CA (US)

(72) Inventors: DaZhi Liu, Davis, CA (US); Bruce Lyeth, Davis, CA (US); Ryan Martin, Elk Grove, CA (US); Kiarash Shahlaie, Sacramento, CA (US)

(73) Assignee: The Regents of the University of California, Oakland, CA (US)

(21) Appl. No.: 18/714,772

(22) PCT Filed: Dec. 6, 2022
(86) PCT No.: PCT/US2022/051931

§ 371 Date: May 30, 2024

Related U.S. Application Data
(60) Provisional application No. 63/287,010, filed on Dec. 7, 2021.

(51) Int. Cl. A61K 31/506; A61K 31/519; A61P 25/28

(52) U.S. Cl. CPC A61K 31/506; A61K 31/519; A61P 25/28

(57) ABSTRACT

The disclosure provides compositions and methods for treating traumatic brain injury (TBI). The compositions comprise CDK4/CDK6 inhibitors. The methods comprise administering a composition comprising a CDK4/CDK6 inhibitor to a subject in an amount sufficient to reduce one or more symptoms of TBI.

This application claims the benefit of priority to U.S. Provisional Application No. 63/287,010, filed Dec. 7, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. R01NS089901 and R01NS114061, awarded by the National Institutes of Health (NIH). The Government has certain rights in the invention.

### BACKGROUND

Traumatic brain injury (TBI) is a leading cause of death and disability¹, with 69 million individuals estimated to suffer this injury from all causes worldwide each year². The global TBI treatment market is expected to grow steadily from $112.6 billion in 2017 to $156.8 billion in 2024, at a compound annual growth rate (CAGR) of 4.8% from 2018 to 2024³. Geographically, North America holds the major share of global TBI treatment market owing to the growing number of TBI patients, and the increasing research and development activity on diagnosis management³. However, there is currently no U.S. Food and Drug Administration (FDA) approved drug for treatment of TBI. This disclosure provides compositions and methods useful for treating TBI.

### BRIEF SUMMARY

The disclosure provides compositions and methods useful for treating TBI. In one aspect, the disclosure provides a method for treating traumatic brain injury (TBI) in a human subject in need thereof, the method comprising administering a composition comprising a therapeutically effective amount of a selective cyclin-dependent kinase 4 (CDK4)/cyclin-dependent kinase 6 (CDK6) inhibitor to the subject, thereby treating TBI in the human subject.

In some embodiments, the selective CDK4/CDK6 inhibitor is selected from the group consisting of abemaciclib (LY2835219), palbociclib (PD-0332991), ribociclib (LEE011), trilaciclib (CAS No. 1374743-00-6), a salt thereof, and a combination thereof.

In some embodiments, the subject is an adolescent or adult human.

In some embodiments, the therapeutically effective amount comprises a dose of the selective CDK4/CDK6 inhibitor of about 20 mg/day to about 100 mg/day. In some embodiments, the inhibitor is administered in one or more doses. In some embodiments, the inhibitor is administered in a single dose. In some embodiments, the single dose comprises about 20 mg to about 100 mg of the inhibitor. In some embodiments, the single dose comprises about 30 mg of the inhibitor.

In some embodiments, the inhibitor is administered during the acute stage of TBI. In some embodiments, the inhibitor is administered within about 0.1 hours to about 9 hours following TBI.

In some embodiments, the TBI is classified as mild, moderate or severe TBI. In some embodiments, the TBI is classified as mild TBI, and the composition is administered to the subject orally. In some embodiments, the TBI is classified as moderate to severe TBI, and the composition is administered to the subject intravenously.

In some embodiments, the composition has decreased side effects compared to a dose of the CDK4/CDK6 inhibitor administered at 100 mg or greater (e.g., 100 mg, 125 mg, 150 mg, 200 mg, 250 mg, 300 mg, 350 mg, 400 mg. 450 mg, 500 mg, 550 mg, 600 mg, or greater) one time per day. In some embodiments, the composition has decreased side effects compared to a dose of the CDK4/CDK6 inhibitor administered at 100 mg or greater (e.g., 100 mg, 125 mg, 150 mg, 200 mg, 250 mg, 300 mg, 350 mg, 400 mg. 450 mg, 500 mg, 550 mg, 600 mg, or greater) two times per day. In some embodiments, the side effects are selected from those in a row of Table 2.

In some embodiments, the composition increases motor function in the subject following TBI as determined by Assessment of Motor and Process Skills (AMPS). In some embodiments, the composition promotes hippocampal neuronal survival following TBI.

In some embodiments, the composition improves cognitive function in the subject as measured by the Montreal Cognitive Assessment, the Overall Test Battery Mean, a latent ability composite score, the Neuropsychological Deficit Score, or a combination thereof.

In some embodiments, administering the composition to the subject results in decreased symptoms of TBI as determined by the Glasgow outcome scale extended (GOSE). In some embodiments, administering the composition to the subject results in a 2 level improvement (e.g. an increase of 2 levels) on the GOSE at 6 months after the TBI occurred.

In some embodiments, the composition results in decreased symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof.

In some embodiments, the composition results in decreased symptoms of TBI-induced brain damage selected from the group consisting of intracranial hemorrhage, brain hematoma, motor deficits, disruption of the blood brain barrier, brain edema, and a combination thereof.

In some embodiments, the composition results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof.

In some embodiments, the composition further comprises a pharmaceutically acceptable carrier or excipient.

In some embodiments, wherein the composition is administered to the subject intravenously or orally.

### BRIEF DESCRIPTION OF THE DRAWINGS

FIG. 1 shows Abemaciclib (3 mg/kg, administered i.v. at 0, 3 hr, or 6 hrs post-TBI) mitigates spatial motor imbalance one day after CCI-TBI in rats. The Y-axis shows biased swinging to contralateral side (%) of TBI at 22-24 hrs after TBI. Each horizontal line represents the mean±standard error. ##P<0.01 vs sham surgery, *P<0.05 vs TBI/vehicle (unpaired t-tests).

FIGS. 2A-2J show immunohistochemistry of NeuN-positive neurons in the CA2-3 region of the hippocampus in rats, and that abemaciclib (3 mg/kg, administered i.v. at 0, 3, or 6 hrs post-TBI) decreases loss of ipsilateral CA2-3 neurons at 24 hrs after TBI. FIGS. 2A and 2B: Sham operation control. FIGS. 2C and 2D: TBI/Vehicle controls. FIGS. 2E and 2F: abemaciclib administered at 0 hr after TBI. FIGS. 2G and 2H: abemaciclib administered at 3 hrs after TBI. FIGS. 2I and 2J: abemaciclib administered at 6 hrs after TBI. Scale bars: 200 μm in A, C, E, G, I; 50 μm in B, D, F, H, J.

FIG. 3 shows that TBI causes loss of neurons in the ipsilateral side of TBI one day after TBI (##P<0.01 vs. sham control), while a single acute administration of abemaciclib (3 mg/kg, administered i.v., at 0, 3, or 6 hrs post-TBI) can significantly promote survival of CA2-3 neurons 24 hrs after TBI. The Y-axis shows cell numbers. Each horizontal line represents the mean±standard error. ##P<0.01 vs sham surgery control; *P<0.05, **P<0.01 (unpaired t-tests).

### DETAILED DESCRIPTION

### Definitions

As used herein, the term “brain injury” refers to a direct or indirect damage to the brain or head. A brain injury may be caused by a direct or indirect physical damage to the brain or head (i.e., a fall, an assault, or a motor vehicle accident). A brain injury may also be caused by a disease that is directly or indirectly related to the brain or head (i.e., meningitis). A brain injury may be confirmed to one area of the brain or head or involve more than one area of the brain or head. Some symptoms of brain injury include, but are not limited to, neuroinflammation, hypotension, hypoxia, edema, abnormalities in glucose utilization, cellular metabolism, membrane fluidity, synaptic function, and structural integrity of the brain. In some embodiments, a brain injury is an acute brain injury, e.g., traumatic brain injury (TBI), concussion, intracerebral hemorrhage (ICH), intraventricular hemorrhage (IVH), subarachnoid hemorrhage (SAH), seizure, and ischemic stroke.

As used herein, the terms “subject,” “individual,” and “patient” are used interchangeably herein to refer to a vertebrate, a mammal, or a human. Mammals include, but are not limited to, rodents, murines, rats, simians, humans, farm animals, sport animals, and pets. Tissues, cells and their progeny of a biological entity obtained in vivo or cultured in vitro are also encompassed.

As used herein, the term “administering” includes oral administration, topical contact, administration as a suppository, intravenous, intraperitoneal, intramuscular, intralesional, intrathecal, intranasal, intraosseous, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arterial, intradermal, subcutaneous, intraperitoneal, intraventricular, intraosseous, and intracranial. Other modes of delivery include, but are not limited to, the use of liposomal formulations, intravenous infusion, transdermal patches, etc. In particular embodiments of the methods described herein, the inhibitor may be administered to the subject intravenously.

As used herein, the term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. Therapeutic benefit can also mean to effect a cure of one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. In particular embodiments, beneficial results that may be obtained from the methods for treating a brain injury in a subject described herein include, e.g., prevention or reduction of lymphocyte infiltration (e.g., T cell infiltration), prevention or reduction of blood-brain barrier (BBB) disruption, and prevention or reduction of neuronal death.

As used herein, the term “therapeutically effective amount” refers to an amount, e.g., pharmaceutical dose, effective in inducing a desired biological effect in a subject or patient or in treating a patient having a condition or disorder described herein. It is also to be understood herein that a “therapeutically effective amount” may be interpreted as an amount giving a desired therapeutic effect, either taken in one dose or in any dosage or route, taken alone or in combination with other therapeutic agents. A therapeutically effective amount may be an amount that treats, prevents, alleviates, abates, or reduces the severity of symptoms of diseases and disorders (e.g., a brain injury).

As used herein, the term “pharmaceutical composition” refers to a medicinal or pharmaceutical formulation that contains an active ingredient as well as one or more excipients and diluents to enable the active ingredient suitable for the method of administration. The pharmaceutical composition of the present disclosure includes pharmaceutically acceptable components that are compatible with an inhibitor of the disclosure. The pharmaceutical composition may be in aqueous form for intravenous or subcutaneous administration or in tablet or capsule form for oral administration.

As used herein, the term “pharmaceutically acceptable carrier” refers to an excipient or diluent in a pharmaceutical composition. The pharmaceutically acceptable carrier must be compatible with the other ingredients of the formulation and not deleterious to the recipient.

The nature of the carrier differs with the mode of administration. For example, for intravenous administration, an aqueous solution carrier is generally used; for oral administration, a solid carrier can be used.

The term “IC₅₀” refers to half-maximal inhibitory concentration, and is the most widely used and informative measure of a drug's efficacy. It indicates how much of a drug or compound is needed to inhibit a biological process by half, thus providing a measure of potency of an antagonist drug. The IC₅₀ of a pharmacological compound can be determined based on assays that utilize whole cell systems or by surface plasmon resonance to accurately determine IC₅₀ values of individual inhibitor-target pairs.

All numerical ranges disclosed herein include the endpoints at each end of the range, unless otherwise excluded, and all numerical values in between the endpoints, to the first significant digit. For example, a range of 1 to 10 can include the values 1.0, 1.1, 1.2, 1.3, . . . 9.7, 9.8, 9.9 and 10.0.

The term “about,” when modifying a numerical value of the disclosure, refers to values that include normal experimental variation in the art, and includes values that are plus or minus 1% to 10% of a recited value, e.g., +/−1%, 2%, 3%, 4%, 5%, 6%, 7%, 8% 9%, or 10%. All numerical values and ranges are understood to be modified by the term “about” regardless of whether or not the term “about” is expressly used to modify the value.

### Introduction

The present disclosure provides compositions and methods for treating traumatic brain injury (TBI). In some embodiments, the compositions comprise inhibitors of cyclin-dependent kinase 4 and cyclin-dependent kinase 6 (referred to as CDK4/6 inhibitors). In some embodiments, the inhibitors selectively inhibit CDK4 and CDK6. In some embodiments, the inhibitors inhibit CDK4 and CDK6 but do not inhibit other cyclin-dependent kinases (CDKs), such as CDK1, CDK2, CDK3, CDK5, CDK7, CDK8, CDK9, CDK11, CDK12, CDK13, CDK19, CLK, or Cdc. In some embodiments, the CDK4/6 inhibitor has an IC₅₀ of less than about 100 nanomolar (nM) in cell free assays. For example, in some embodiments, the CDK4/6 inhibitor has an IC₅₀ of less than about 100, 90, 80, 70, 60, 50, 40, 30, 20, or less than about 10 nM in cell free assays. In some embodiments, the CDK4/6 inhibitor has an IC₅₀ of about 1 nM to about 40 nM in cell free assays. In some embodiments, the CDK4/6 inhibitor has an IC₅₀ of 1 nM, 2 nM, 3 nM, 4 nM, 5 nM, 6 nM, 7 nM, 8 nM, 9 nM, 10 nM, 11 nM, 12 nM, 13 nM, 14 nM, 15 nM, 16 nM, 17 nM, 18 nM, 19 nM, 20 nM, 25 nM, 30 nM, 35 nM, 39 nM, or 40 nM in cell free assays. The IC₅₀ and doses for representative CDK4/CDK6 inhibitors are shown in Table 1 below.

TABLE 1
IC₅₀ and doses for representative CDK4/CDK6 inhibitors useful treat TBI.

| | | | Estimated | HUMAN equivalent | |
|---|---|---|---|---|---|
| | | | dose in | dose (HED) | |
| | | | vivo for | caculated from rat | |
| | | in vitro | rats, | in vivo data, | |
| | | IC50 (nM) | according | according to body | |
| Target | Drug/CDK4/6 | in cell-free | to the in | surface area-based | FDA-approved doses for |
| kianse | inhibitor | assays | vitro IC50 | practice guide | cancer treatments |
| CDKs | abemaciclib | 2 nM for | 1-10 | 29.16 mg (for a 60 kg | Recommended starting dose |
| | | CDK4; | mg/kg | adult) | in combination with |
| | | 10 nM for | | | fulvestrant or an aromatase |
| | | CDK6 | | | inhibitor: 150 mg twice |
| | | | | | daily; Recommended |
| | | | | | starting dose as |
| | | | | | monotherapy: 200 mg twice |
| | | | | | daily. |
| | palbociclib | 11 nM for | 6-60 | ND | 125 mg orally once daily for |
| | | CDK4; | mg/kg | | 21 days followed by 7 days |
| | | 16 nM for | | | off treatment. |
| | | CDK6 | | | |
| | ribociclib | 10 nM for | 5-50 | ND | 600 mg orally once daily for |
| | | CDK4; | mg/kg | | 21 days followed by 7 days |
| | | 39 nM for | | | off treatment. |
| | | CDK6 | | | |
| | trilaciclib | 1 nM for | 0.5-5 | ND | 388.8 mg/day/adult (for a 60 |
| | | CDK4; | mg/kg | | kg adult) as a 30-minute |
| | | 4 nM for | | | intravenous infusion |
| | | CDK6 | | | completed within 4 hours |
| | | | | | prior to the start of |
| | | | | | chemotherapy on each day |
| | | | | | chemotherapy is |
| | | | | | administered. |
| | lerociclib | 1 nM for | 0.5-5 | ND | NA |
| | (G1T38) | CDK4; | mg/kg | | |
| | | 2 nM for | | | |
| | | CDK6 | | | |
| | ON123300 | 3.9 nM for | 2-20 | ND | NA |
| | | CDK4; ND | mg/kg | | |
| | | for CDK6. | | | |

ND = Not Determined.
NA = Not Applicable (not FDA approved).

In some embodiments, the inhibitor is administered to a human subject suffering from TBI. While CDK4/6 inhibitors have been approved by the FDA to treat cancer and tumors, CDK4/6 inhibitors have not been approved by the FDA for treating TBI. In addition, the FDA has approved the administration of CDK4/6 inhibitors to human cancer patients at relatively high doses, for example 125 to 600 milligrams (mg)/day to an adult human. However, there are no reports of treating TBI with relatively low doses of CDK4/6 inhibitors, such as less than about 100 mg per day.

The compositions and methods of the disclosure provide advantages over existing treatments for TBI. For example, chronic administration of CDK4/6 inhibitors at higher doses typically administered for treating cancer (e.g., starting doses equal to or greater than 125 mg/day for 21 days) can result in undesirable side effects, such as diarrhea, nausea, abdominal pain, infections, tiredness, anemia, leukopenia, decreased or loss of appetite, vomiting, headache, thrombocytopenia, alopecia, hypoxia, dyspnea, sores on the lips, mouth, or throat, weight loss, hair loss, itching, rash, changes in taste, dizziness, joint pain, pain in the upper right part of the stomach, yellowing of the skin or eyes, bleeding or bruising easily, pain in arms or legs, swelling of the hands, feet, legs or ankles, shortness of breath, chest pain, rapid breathing, fast, irregular, or pounding heartbeat, fever, chills, cough or other signs of infection, or pale skin. See the internet at medlineplus.gov/druginfo/meds/a617049.html. Common side effects associated with FDA approved doses of representative CDK4/6 inhibitors are shown in Table 2 below.

TABLE 2
Common side effects associated with FDA approved
doses of representative CDK4/6 inhibitors.

| Drug/CDK4/6 | |
|---|---|
| inhibitor | Common Side effects at FDA-approved doses (Medline Plus) |
| abemaciclib | diarrhea, nausea, abdominal pain, infections, tiredness, anemia, leukopenia, |
| | decreased or loss of appetite, vomiting, headache, thrombocytopenia, alopecia, |
| | hypoxia, dyspnea, sores on the lips, mouth, or throat, weight loss, hair loss, |
| | itching, rash, changes in taste, dizziness, joint pain, pain in the upper right part |
| | of the stomach, yellowing of the skin or eyes, bleeding or bruising easily, pain |
| | in arms or legs, swelling of the hands, feet, legs or ankles, shortness of breath, |
| | chest pain, rapid breathing, fast, irregular, or pounding heartbeat, fever, chills, |
| | cough or other signs of infection, pale skin. |
| palbociclib | nausea; diarrhea; vomiting; decreased appetite; changes in taste; tiredness; |
| | numbness or tingling in your arms, hands, legs, and feet; sores on the lips, |
| | mouth, or throat; unusual hair thinning or hair loss; dry skin; rashfever, chills, |
| | or signs of infection; shortness of breath; dizziness; fast, irregular, or pounding |
| | heartbeat; weakness; unusual bleeding or bruising; nosebleed. |
| ribociclib | diarrhea; constipation; stomach pain; headache; hair loss; back pain; itching; |
| | mouth sores; swelling of the hands, feet, ankles, or lower legs; difficulty falling |
| | asleep or staying asleep; fever, sore throat, chills, or other signs of infection; |
| | rash; blistering or peeling of the skin; burning or blistering of the lips, eyes, or |
| | mouth; rapid, irregular, or pounding heartbeat; difficulty breathing or shortness |
| | of breath; cough with or without mucus; or chest pain; dizziness; fainting; |
| | yellowing of the skin or eyes; dark or brown (tea-colored) urine; tiredness; loss |
| | of appetite; pain in upper right side of stomach; nausea; vomiting; bleeding or |
| | bruising more easily than normal. |
| trilaciclib | tiredness; headache; upper right stomach pain; fever, cough, shortness of |
| | breath, or other signs of infection; injection site pain, swelling, redness, |
| | warmth, or itching; red, hot, swollen area on the skin; facial, eye, and tongue |
| | swelling, difficulty breathing or swallowing; rash; itching; hives. |

In addition, CDK4/6 inhibitors are typically administered to cancer patients chronically (over time such as days, weeks or months), which can cause long-term inhibition of kinases (e.g., CDK4 and CDK6). Long term inhibition of kinases can result in additional undesirable side effects, as these kinases play important roles in neurogenesis, blood-brain barrier self-repair, and other important cellular processes during the recovery phase after TBI. Therefore, the compositions described herein can be administered as a single dose during the acute phase of TBI, rather than chronically over time, which should further reduce the undesirable side effects associated with chronic administration of CDK4/6 inhibitors.

### Methods

The methods of the disclosure are useful for treating TBI in a subject, such as a human. In some embodiments, the methods comprise administering a therapeutically effective amount of a composition comprising a CDK4/6 inhibitor to the subject. In some embodiments, the subject is an adolescent or adult human. The CDK4/6 inhibitor can be administered at a dose of less than about 100 mg/day, e.g. at a dose of less than 100 mg, less than 90 mg, less than 80 mg, less than 70 mg, less than 60 mg, less than 50 mg, less than 40 mg, less than 30 mg, less than 20 mg or less than 10 mg/day. In some embodiments, the CDK4/6 inhibitor can be administered at a dose of about 10 mg/day to about 100 mg/day, about 10 mg/day to about 90 mg/day, about 10 mg/day to about 80 mg/day, about 10 mg/day to about 70 mg/day, about 10 mg/day to about 60 mg/day, about 10 mg/day to about 50 mg/day, about 10 mg/day to about 40 mg/day, about 10 mg/day to about 30 mg/day, about 10 mg/day to about 20 mg/day, about 20 mg/day to about 100 mg/day, about 20 mg/day to about 90 mg/day, about 20 mg/day to about 80 mg/day, about 20 mg/day to about 70 mg/day, about 20 mg/day to about 60 mg/day, about 20 mg/day to about 50 mg/day, about 20 mg/day to about 40 mg/day, about 20 mg/day to about 30 mg/day, about 30 mg/day to about 100 mg/day, about 30 mg/day to about 90 mg/day, about 30 mg/day to about 80 mg/day, about 30 mg/day to about 70 mg/day, about 30 mg/day to about 60 mg/day, about 30 mg/day to about 50 mg/day, or about 30 mg/day to about 40 mg/day.

In some embodiments, the CDK4/6 inhibitor can be administered at a dose of about 20 to about 40 mg/day, e.g. at a dose of about 20, 21, 22, 23, 24, 25, 26, 27, 28, 29, 30, 31, 32, 33, 34, 35, 36, 37, 38, 39 or 40 mg/day to an adolescent or adult human. In some embodiments, the inhibitor can be administered as a single dose of about 20 to about 40 mg, e.g., a single dose of about 20, 21, 22, 23, 24, 25, 26, 27, 28, 29, 30, 31, 32, 33, 34, 35, 36, 37, 38, 39 or 40 mg to an adolescent or adult human. The dose administered is typically determined based on the weight of the subject, and the values provided herein are based on a 60 kg human. One of ordinary skill in the art can adjust the dose based on the body surface area, weight or mass of an individual subject. See, e.g., Nair A B, Jacob S. A simple practice guide for dose conversion between animals and human. J Basic Clin Pharm. 2016; 7 (2): 27-31.

The cyclin-dependent kinases (CDKs) are a large family of heterodimeric serine/threonine protein kinases that coordinate cell cycle progression through phosphorylation of well-defined enzymatic and structural targets. CDKs phosphorylate a broad range of proteins that initiate and regulate the various events that characterize each phase of the cell cycle (see Grana and Reddy, “Cell cycle control in mammalian cells: role of cyclins, cyclin dependent kinases (CDKs), growth suppressor genes and cyclin-dependent kinase inhibitors (CKIs).” Oncogene. 1995 Jul. 20; 11 (2): 211-9. PMID: 7624138.). Based on their functions, CDKs can be divided into two main sub-groups: cell cycle CDKs (such as CDK1, CDK2, CDK4, CDK6) and transcriptional CDKs (such as CDK7, CDK8, and CDK9).

In some embodiments, the CDK4/6 inhibitor is selected from abemaciclib (LY2835219), palbociclib (PD-0332991), ribociclib (LEE011), trilaciclib (CAS No. 1374743-00-6), or a salt thereof. In some embodiments, the CDK4/6 inhibitor is selected from one or more of, or a combination of abemaciclib (LY2835219), palbociclib (PD-0332991), ribociclib (LEE011), trilaciclib (CAS No. 1374743-00-6; G1T28), or a salt thereof. In some embodiments, the CDK4/6 inhibitor is selected from abemaciclib mesylate, palbociclib HCL, palbociclib isethionate, ribociclib succinate, or ribociclib hydrochloride, or combinations thereof.

In some embodiments, the CDK4/6 inhibitor is an ATP-competitive inhibitor. In some embodiments, the ATP-competitive inhibitor is selected from abemaciclib (LY2835219), palbociclib (PD-0332991), ribociclib (LEE011) or trilaciclib (G1T28).

In some embodiments, the CDK4/6 inhibitor is lerociclib (G1T38) (see the internet at selleckchem.com/products/g1t38.html) or ON123300 (see the internet at selleckchem.com/products/on123300.html).

Representative CDK4/6 inhibitors useful in the methods of the disclosure are described in U.S. Pat. No. 7,855,211 (abemaciclib; Eli Lilly and Company), U.S. Pat. Nos. 6,936,612, 7,208,489 and 7,456,168, 7,345,171, 7,863,278 and 10,723,730 (palbociclib; Pfizer Inc.), U.S. Pat. Nos. 8,324,225, 8,415,355, 8,685,980, 8,962,630, 9,416, 136, 9,193,732, 9,868,739, and 10,799,506 (ribociclib; Novartis AG), and U.S. Pat. Nos. 8,691,830, 8,829,012, 9,102,682, 9,487,530, 9,499,564, 9,957,276, 10,189,851, 10,696,682, 10,085,992, 10,927,120, 10,966,984, 11,040,042 (trilaciclib; G1 Therapeutics, Inc.), which are incorporated by reference herein.

In some embodiments, the subject is a human. In some embodiments, the subject is an adult human. In some embodiments, the subject is an adolescent human.

In some embodiments, the subject is an adolescent or adult human, and the dose of the CDK4/6 inhibitor comprises less than 100 mg/day, e.g. a dose of less than 100 mg, less than 90 mg, less than 80 mg, less than 70 mg, less than 60 mg, less than 50 mg, less than 40 mg, less than 30 mg, less than 20 mg or less than 10 mg/day. In some embodiments, the subject is an adolescent or adult human, and the dose of the CDK4/6 inhibitor comprises about 20 mg/day to about 100 mg/day (e.g., a dose of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99, or 100 mg/day to an adolescent or adult human). In some embodiments, the inhibitor can be administered as a single dose of about 20 to about 100 mg to an adolescent or adult human (e.g., a single dose of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99, or 100 mg to an adolescent or adult human). In some embodiments, the subject is an adolescent or adult human, and the dose of the CDK4/6 inhibitor comprises about 30 mg/day. In some embodiments, the composition comprising the inhibitor is administered in one or more doses (e.g., one, two, three, four, five or more doses). Thus, in embodiments where the subject is an adolescent or adult human, the composition can be administered in one or more doses (e.g., one, two, three, four, five or more doses) comprising a total dose of about 20 mg/day to about 100 mg/day of a CDK4/6 inhibitor (e.g., a total dose of a CDK4/6 inhibitor of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99, or 100 mg/day). In some embodiments, the subject is an adolescent or adult human, and the composition is administered in one or more doses comprising a total dose of about 30 mg/day of a CDK4/6 inhibitor.

The composition comprising a CDK4/6 inhibitor can be administered one or more times per day, daily, weekly, or monthly. In some embodiments, the composition is administered one time during the acute phase of TBI. In some embodiments, the composition is administered multiple times during the chronic phase of TBI.

In some embodiments, the inhibitor is palbociclib (PD-0332991), and the dose for treating TBI in human subjects can range from about 20 mg/day to about 100 mg/day (e.g., a dose of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99, or 100 mg/day) for an adolescent or adult human. In some embodiments, the inhibitor is ribociclib (LEE011), and the dose for treating TBI in human subjects can range from about 20 mg/day to about 100 mg/day (e.g., a dose of about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99, or 100 mg/day) for an adolescent or adult human. Note that the highest doses of palbociclib and ribociclib for treating TBI are still lower than the FDA approved doses for cancer treatments.

In some embodiments, the composition is administered in a single dose. In some embodiments, the composition is administered in a single dose during the acute phase of TBI. In some embodiments, the composition is administered in a single dose between about 0.1 to about 9 hours following TBI (e.g., about 0.1, 0.2, 0.3, 0.4, 0.5, 1, 2, 3, 4, 5, 6, 7, 8, or 9 hours following TBI). In some embodiments, the composition is administered in a single dose comprising about 20 mg to about 40 mg of a CDK4/6 inhibitor (e.g., about 20, 21, 22, 23, 24, 25, 26, 27, 28, 29, 30, 31, 32, 33, 34, 35, 36, 37, 38, 39 or 40 mg of a CDK4/6 inhibitor) between about 0.1 hour to about 9 hours following TBI. In some embodiments, the composition is administered in a single dose comprising about 20 mg of a CDK4/6 inhibitor between about 0.1 hour to about 9 hours following TBI.

The TBI amendable to treatment by the methods of the disclosure can be classified as mild, moderate or severe TBI. In some embodiments, the TBI is classified as mild TBI (e.g., the subject has not lost consciousness or has regained consciousness), and the composition is administered to the subject orally, for example as a pill or tablet. The Table below shows a representative classification of TBI severity.

TABLE
Classification of TBI Severity.

| Criteria | Mild | Moderate | Severe |
|---|---|---|---|
| Structural imaging | Normal | Normal or | Abnormal |
| | | abnormal | |
| Loss of Consciousness (LOC) | 0-30 min | >30 min | >24 hrs |
| | | and <24 hrs | |
| Alteration of consciousness/ | Less than or | >24 hours. Severity based | |
| mental state (AOC) | equal to 24 | on other criteria | |
| | hrs | | |
| Post-traumatic amnesia (PTA) | 0-1 day | >1 and <7 | >7 days |
| | | days | |
| Glasgow Coma Scale (best | 13-15 | 9-12 | <9 |
| available score in first 24 | | | |
| hours) | | | |

From: APPENDIX C, DEFINITION OF MTBI FROM THE VA/DOD CLINICAL PRACTICE GUIDELINE FOR MANAGEMENT OF CONCUSSION/MILD TRAUMATIC BRAIN INJURY (2009)

As noted above, the compositions of the disclosure can be administered at CDK4/6 inhibitor doses that reduce the side effects associated with doses that are approved for treatment of cancer patients. Thus, in some embodiments, the composition has decreased side effects compared to a dose of inhibitor administered to a subject at greater than 100 mg/day. In some embodiments, the inhibitor is selected from abemaciclib (LY2835219), palbociclib (PD-0332991), ribociclib (LEE011), trilaciclib (CAS No. 1374743-00-6), or a salt thereof, and the composition has decreased side effects compared to a dose of the same inhibitor administered to a subject at greater than 100 mg/day.

In some embodiments, the compositions of the disclosure are administered at doses that reduce a side effect (e.g., a single side effect, one or more side effects, a plurality of side effects, or all the side effects) associated with a CDK4/CDK6 inhibitor shown in Table 5 compared to the dose of a CDK4/6 inhibitor approved for treatment of cancer patients. In some embodiments, the compositions of the disclosure are administered at doses that reduce a side effect (e.g., a single side effect, one or more side effects, a plurality of side effects, or all the side effects) associated with a CDK4/CDK6 inhibitor shown in Table 5 compared to the dose of the same CDK4/6 inhibitor approved for treatment of cancer patients. In some embodiments, the compositions of the disclosure are administered at doses that are lower than the FDA approved doses for the same CDK4/6 inhibitor, and reduce a side effect (e.g., a single side effect, one or more side effects, a plurality of side effects, or all the side effects) associated with the CDK4/CDK6 inhibitor shown in Table 5.

In some embodiments, the side effects are selected from the group consisting of diarrhea, nausea, abdominal pain, infections, tiredness, anemia, leukopenia, decreased or loss of appetite, vomiting, headache, thrombocytopenia, alopecia, hypoxia, dyspnea, sores on the lips, mouth, or throat, weight loss, hair loss, itching, rash, changes in taste, dizziness, joint pain, pain in the upper right part of the stomach, yellowing of the skin or eyes, bleeding or bruising easily, pain in arms or legs, swelling of the hands, feet, legs or ankles, shortness of breath, chest pain, rapid breathing, fast, irregular, or pounding heartbeat, fever, chills, cough or other signs of infection, pale skin and a combination thereof.

In some embodiments, the composition improves cognitive function in the subject as measured by the Montreal Cognitive Assessment, the Overall Test Battery Mean, a latent ability composite score, the Neuropsychological Deficit Score, or a combination thereof. See Silverberg N D, et al., Developing a Cognition Endpoint for Traumatic Brain Injury Clinical Trials. J Neurotrauma. 2017 Jan. 15; 34 (2): 363-371; Mishra K., et al. Montreal cognitive Assessment Score: A Screening Tool for Cognitive Function in Traumatic Brain Injury (TBI) Population. Journal of Neurology & Neuromedicine, 2020.

In some embodiments, administering the composition to the subject results in decreased symptoms of TBI as determined by the Glasgow outcome scale extended (GOSE). The Glasgow Outcome Scale (GOS) is a global scale for functional outcome that rates patient status into one of five categories: Dead, Vegetative State, Severe Disability, Moderate Disability or Good Recovery. The Extended GOS (GOSE) provides more detailed categorization into eight categories by subdividing the categories of severe disability, moderate disability and good recovery into a lower and upper category, as shown in Table 3 below (see Jennett B, Bond M: Assessment of outcome after severe brain damage. Lancet 1:480-484, 1975; Teasdale G M, Pettigrew L E, Wilson J T, Murray G, Jennett B. Analyzing outcome of treatment of severe head injury: A review and update on advancing the use of the Glasgow Outcome Scale. Journal of Neurotrauma 1998; 15:587-597; Wilson J T L, Pettigrew L E L, Teasdale G M. Structured interviews for the Glasgow Outcome Scale and the Extended Glasgow Outcome Scale: Guidelines for Their Use. J Neurotrauma 15 (8): 573-85. 1997; Wilson J T, Slieker F J, Legrand V, Murray G, Stocchetti N, Maas AI. Observer variation in the assessment of outcome in traumatic brain injury: experience from a multicenter, international randomized clinical trial. Neurosurgery. Jul; 61 (1): 123-8; discussion 128-9. 2007; Sander, A. (2002). The Extended Glasgow Outcome Scale. The Center for Outcome Measurement in Brain Injury. See the internet at tbims.org/combi/gose)

TABLE 3
Extended Glasgow Outcome Scale (GOSE)

| | | |
|---|---|---|
| 1. | Death (D) | |
| 2. | Vegetative | (VS) Condition of unawareness with only |
| | State (VS) | reflex responses but with periods of |
| | | spontaneous eye opening. |
| 3. | Low Severe | Patient who is dependent for daily support for |
| | Disability | mental or physical disability, usually a |
| | (SD−) | combination of both. If the patient can be left |
| | | alone for more than 8 h at home it is upper |
| | | level of SD, if not then it is low level of SD. |
| 4. | Upper Severe | |
| | Disability | |
| | (SD+) | |
| 5. | Low Moderate | Patients have some disability such as aphasia, |
| | Disability | hemiparesis or epilepsy and/or deficits of |
| | (MD−) | memory or personality but are able to look |
| | | after themselves. They are independent at |
| | | home but dependent outside. If they are able |
| | | to return to work even with special |
| | | arrangement it is upper level of MD, if not |
| | | then it is low level of MD |
| 6. | Upper Moderate | |
| | Disability | |
| | (MD+) | |
| 7. | Low Good | Resumption of normal life with the capacity to |
| | Recovery | work even if pre-injury status has not been |
| | (GR−) | achieved. Some patients have minor |
| | | neurological or psychological deficits. If these |
| | | deficits are not disabling then it is upper level |
| | | of GR, if disabling then it is lower level of |
| | | GR. |
| 8. | Upper Good | |
| | Recovery | |
| | (GR+) | |

Use of the structured interview is recommended to facilitate consistency in ratings.

In some embodiments, administering the composition to the subject results in at least a 2 level improvement on the GOSE at 6 months after the TBI occurred. For example, administering the composition to the subject can result in an increase from level 2 (Vegetative State) to level 4 (Upper Severe Disability (SD+)); an increase from level 3 (Low Severe Disability (SD−)) to level 5 (Low Moderate Disability (MD−)); an increase from level 4 (Upper Severe Disability (SD+)) to level 6 (Upper Moderate Disability (MD+)); an increase from level 5 (Low Moderate Disability (MD−)) to level 7 (Low Good Recovery (GR−); or an increase from level 6 (Upper Moderate Disability (MD+)) to level 8 (Upper Good Recovery (GR+)) on the GOSE at 6 months after the TBI occurred.

In some embodiments, administering the composition to the subject results in a 2 level, 3 level or 4 level improvement on the GOSE at 6 months after the TBI occurred. For example, administering the composition to the subject can result in an increase from level 2 (Vegetative State) to level 4 (Upper Severe Disability (SD+)); an increase from level 2 to level 5 (Low Moderate Disability (MD−)); or an increase from level 2 to level 6 (Upper Moderate Disability (MD+)) on the GOSE at 6 months following the TBI. In another example, administering the composition to the subject can result in an increase from level 3 ((Low Severe Disability (SD−)) to level 5 (Low Moderate Disability (MD−)); an increase from level 3 to level 6 (Upper Moderate Disability (MD+)); or an increase from level 3 to level 7 (Low Good Recovery (GR−) on the GOSE at 6 months following the TBI. In another example, administering the composition to the subject can result in an increase from level 4 (Upper Severe Disability (SD+)) to level 6 Upper Moderate Disability (MD+); an increase from level 4 to level 7 (Low Good Recovery (GR−); or an increase from level 4 to level 8 (Upper Good Recovery (GR+)) on the GOSE at 6 months following the TBI.

In some embodiments, administering the composition to the subject results in a 2 level, 3 level, 4 level, 5 level or 6 level improvement on the GOSE at 6 months after TBI.

In some embodiments, the method results in a decrease in one or more symptoms of TBI. In some embodiments, administering a composition comprising a CDK4/6 inhibitor to the subject results in a decrease in one or more symptoms of TBI. In some embodiments, administering a composition comprising a CDK4/6 inhibitor to the subject comprising a dose of CDK4/6 inhibitor less than about 100 mg/day (e.g., a dose of CDK4/6 inhibitor less than 100 mg, less than 90 mg, less than 80 mg, less than 70 mg, less than 60 mg, less than 50 mg, less than 40 mg, less than 30 mg, less than 20 mg or less than 10 mg/day) results in a decrease in one or more symptoms of TBI. In some embodiments, the subject is an adolescent or adult human, and administering a composition comprising a CDK4/6 inhibitor to the subject in one or more doses comprising a dose of inhibitor of about 20 mg/day to about 40 mg/day (e.g., a dose of CDK4/6 inhibitor of about 20, 21, 22, 23, 24, 25, 26, 27, 28, 29, 30, 31, 32, 33, 34, 35, 36, 37, 38, 39 or 40 mg/day) results in a decrease in one or more symptoms of TBI. In some embodiments, the subject is an adolescent or adult human, and administering a composition comprising a CDK4/6 inhibitor to the subject in one or more doses comprising a dose of inhibitor of about 20 mg/day results in a decrease in one or more symptoms of TBI. In some embodiments, the subject is an adolescent or adult human, and administering a single dose of a composition comprising a CDK4/6 inhibitor to the subject results in a decrease in one or more symptoms of TBI. In some embodiments, the single dose administered to the subject comprises about 20 mg to about 40 mg of a CDK4/6 inhibitor (e.g., a single dose of CDK4/6 inhibitor of about 20, 21, 22, 23, 24, 25, 26, 27, 28, 29, 30, 31, 32, 33, 34, 35, 36, 37, 38, 39 or 40 mg) results in a decrease in one or more symptoms of TBI. In some embodiments, the subject is an adolescent or adult human, and administering a single dose of a composition comprising about 30 mg of a CDK4/6 inhibitor to the subject results in a decrease in one or more symptoms of TBI. In some embodiments, the inhibitor is selected from abemaciclib (LY2835219), palbociclib (PD-0332991), ribociclib (LEE011), or trilaciclib (CAS No. 1374743-00-6), a salt thereof, or combinations thereof.

In some embodiments, administering a composition of the disclosure to a subject results in a decrease in one or more symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof. In some embodiments, administering a composition of the disclosure to a subject results in a decrease in one or more symptoms of TBI selected from the group consisting of intracranial hemorrhage, brain hematoma, motor deficits, disruption of the blood brain barrier, brain edema, and a combination thereof.

In some embodiments, administering a composition of the disclosure to a subject results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof. Primary TBI refers to injury that occurs at the moment of initial trauma, and includes: skull fracture (breaking of the bony skull), contusions (bruise/bleed on the brain) that can lead to hematomas (blood clots in the meningeal layers or in the cortical/subcortical structures as a result of the trauma), concussions (low velocity injury resulting in functional deficits without pathological injury), lacerations (tears in brain tissue or blood vessels of the brain), diffuse axonal injury (traumatic shearing forces leading to tearing of nerve fibers in the white matter tracts).

Primary injuries can be caused by either a penetrating (open-head) injury or a nonpenetrating (closed-head) injury. A penetrating (open-head) injury involves an open wound to the head from a foreign object (e.g., bullet). It is typically marked by focal damage that occurs along the route the object has traveled in the brain that includes fractured/perforated skull, torn meninges, and damage to the brain tissue. A nonpenetrating (closed-head) injury is marked by brain damage due to indirect impact without the entry of any foreign object into the brain. The skull may or may not be damaged, but there is no penetration of the meninges. Nonpenetrating injuries can be of two types: acceleration and non-acceleration injuries. Acceleration injuries are caused by movement of the brain within the unrestrained head (e.g. whiplash injury). If the force impacting the head is strong enough, it can cause a contusion at the site of impact and the opposite side of the skull, causing an additional contusion (coup-contrecoup injury). Non-acceleration injuries are caused by injury to a restrained head and, therefore, no acceleration or deceleration of the brain occurs within the skull (e.g., blow to the head). These usually result in deformation (fracture) of the skull, causing focal localized damage to the meninges and brain.

Secondary injury occurs as an indirect result of the insult. It results from processes initiated by the initial trauma and typically evolves over time. Secondary injury includes ischemia (insufficient blood flow); hypoxia (insufficient oxygen in the brain); hypo/hypertension (low/high blood pressure); cerebral edema (swelling of the brain); raised intracranial pressure (increased pressure within the skull), which can lead to herniation (parts of the brain are displaced); hypercapnia (excessive carbon dioxide levels in the blood); meningitis (infection of the meningeal layers) and brain abscess; biochemical changes (changes in levels of neurotransmitters, sodium, potassium, etc.); epilepsy. See Hegde, M. N. (2006). A coursebook on aphasia and other neurogenic language disorders (3rd ed.). Clifton Park, NY: Delmar Cengage Learning.

### Pharmaceutical Compositions

The compositions of the disclosure include pharmaceutical compositions. The pharmaceutical compositions can include a CDK4/6 inhibitor of the disclosure. In some embodiments, the inhibitor is a CDK4/6 inhibitor selected from abemaciclib (LY2835219), palbociclib (PD-0332991), ribociclib (LEE011), trilaciclib (CAS No. 1374743-00-6), a salt thereof, or combinations thereof.

In some embodiments, the pharmaceutical composition further comprises an excipient or pharmaceutically acceptable carrier. Pharmaceutically acceptable carriers and formulations are described in REMINGTON'S PHARMACEUTICAL SCIENCES, 23RD ED., Academic Press (2020). The pharmaceutical compositions may be administered as a formulation prepared in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, or polysorbate 80, or as solid formulations in appropriate excipients.

In some embodiments, a therapeutically effective amount of the pharmaceutical composition is administered to the subject in an amount sufficient to treat the brain injury (e.g., TBI), prevent or reduce neuronal death, prevent or reduce lymphocyte infiltration (e.g., T cell infiltration), and/or prevent or reduce BBB disruption after the brain injury (e.g., TBI).

In some embodiments, a therapeutically effective amount of the pharmaceutical composition comprises a dose of CDK4/6 inhibitor less than about 100 mg/day, e.g., a dose of CDK4/6 inhibitor less than 100 mg, less than 90 mg, less than 80 mg, less than 70 mg, less than 60 mg, less than 50 mg, less than 40 mg, less than 30 mg, less than 20 mg or less than 10 mg/day. In some embodiments, a therapeutically effective amount of the pharmaceutical composition comprises a dose of CDK4/6 inhibitor of about 20 mg/day to about 40 mg/day (e.g., about 20, 21, 22, 23, 24, 25, 26, 27, 28, 29, 30, 31, 32, 33, 34, 35, 36, 37, 38, 39 or 40 mg/day). In some embodiments, a therapeutically effective amount of the pharmaceutical composition comprises a dose of CDK4/6 inhibitor of about 30 mg/day.

In some embodiments, a therapeutically effective amount of the pharmaceutical composition comprises a single dose of a CDK4/6 inhibitor less than about 100 mg, e.g., a single dose of CDK4/6 inhibitor less than 100 mg, less than 90 mg, less than 80 mg, less than 70 mg, less than 60 mg, less than 50 mg, less than 40 mg, less than 30 mg, less than 20 mg or less than 10 mg. In some embodiments, a therapeutically effective amount of the pharmaceutical composition comprises a single dose of a CDK4/6 inhibitor of about 20 mg to about 40 mg (e.g., a single dose of a CDK4/6 inhibitor of about 20, 21, 22, 23, 24, 25, 26, 27, 28, 29, 30, 31, 32, 33, 34, 35, 36, 37, 38, 39 or 40 mg). In some embodiments, a therapeutically effective amount of the pharmaceutical composition comprises a single dose of a CDK4/6 inhibitor of about 20 mg. Determination of a therapeutically effective amount is within the capability of those skilled in the art.

### Routes and Administration

Pharmaceutical compositions of the disclosure comprising CDK4/6 inhibitors can be formulated for parenteral administration, e.g., intravenous administration, subcutaneous administration, intramuscular administration, intraarterial administration, intrathecal administration, or intraperitoneal administration. In particular embodiments, the pharmaceutical composition may be formulated for oral administration, for example, as a tablet or pill. For injectable formulations, various effective pharmaceutical carriers are known in the art, see, e.g., ASHP Handbook on Injectable Drugs, Trissel, 18th ed. (2014). Other administration routes include, but are not limited to intravenous, rectal, transmucosal, intestinal, enteral, topical, suppository, through inhalation, intranasal, and intraocular administration.

In some embodiments, the CDK4/6 inhibitor is hydrophilic and can be administered intravenously, for example, to a subject having moderate to severe TBI who is unconsciousness and cannot swallow a pill or tablet. In some embodiments, the hydrophilic CDK4/6 inhibitor is abemaciclib (LY2835219) or palbociclib (PD-0332991), or a salt thereof. In some embodiments, the CDK4/6 inhibitor is lipophilic and can be administered orally to a subject, for example a subject having mild TBI or a subject having TBI who is able to swallow a pill or tablet.

Access of the compositions to the site of brain injury does not depend on the ability of the CDK4/6 inhibitors of the disclosure to cross the blood-brain barrier (BBB). For example, CDK4/6 inhibitors, including hydrophilic (e.g., abemaciclib) and lipophilic (e.g., ribociclib) compounds are able to cross the intact BBB in cases of mild TBI. In addition, CDK inhibitors can reach the brain injury site through broken blood vessels in cases of moderate and severe TBI. Further, CDK4/6 inhibitors of the disclosure can block systemic inflammation and decrease leukocyte infiltration into the brain for the treatment of TBI, even if the CDK4/6 inhibitors do not appear in the brain after TBI.

In some embodiments, administration may include a single dose or multiple doses. In some embodiments, pharmaceutical compositions for injection are presented in unit dosage form, e.g., in ampoules or in multi-dose containers. In some embodiments, a pharmaceutical composition described herein is administered in the form of a dosage unit (e.g., bolus).

The pharmaceutical compositions can be administered in a manner compatible with the dosage formulation and in such amount as is therapeutically effective to result in an improvement or remediation of the symptoms of TBI. In some embodiments, the dose is administered at intervals ranging from more than once per day, once per day, once per week, twice per week, three times per week, four times per week, five times per week, six times per week, once per month to once per three months, for as long as needed to sustain the desired effect. The timing between administrations may decrease as the medical condition improves or increase as the health of the patient declines. The dosage may be adapted by the physician in accordance with conventional factors such as the extent of the disease and different parameters of the subject.

### Kits

In another aspect, the disclosure provides a kit comprising a CDK4/6 inhibitor of the disclosure. The kit can also include one or more separate containers comprising a CDK4/6 inhibitor of the disclosure, along with written or computer readable instructions for administering the CDK4/6 inhibitor to a subject having TBI. In some embodiments, the kit comprises a CDK4/6 inhibitor selected from abemaciclib (LY2835219), palbociclib (PD-0332991), ribociclib (LEE011), trilaciclib (CAS No. 1374743-00-6), a salt thereof, or combinations thereof.

### EXAMPLES

### Example 1

This example describes that a single treatment with a representative CDK4/CDK6 inhibitor improves motor function in a representative experimental model of TBI.

### Methods

Experimental TBI Model-controlled cortical impact (CCI)-induced TBI.

CCI was used to produce TBI in adult male Sprague-Dawley rats (weighing 300-350 grams). The rats were anesthetized using isoflurane (inhalation dose: 4-5% for induction; 1-2% for maintenance) and mounted in a stereotaxic frame. After a scalp incision made along the midline, and a craniotomy (6 mm diameter) was performed with a trephine on the right parietal bone (for rats, the trephine is centered at 4.5 mm posterior to bregma and 3.0 mm lateral to the sagittal suture). Care was taken to ensure that the dura remained intact. Brain injury was induced by the CCI device that consists of a pressure gauge and a stimulus generator-controlled piston. We used impact velocity (4.0 m/s), impact depth (2.5 mm), and impact dwell time (100 ms) to produce moderate TBI. The animals were allowed to recover in an incubator maintained at 37° C. after closing the scalp using 4-0 suture over the craniotomy. Assessment of the righting reflex was done by placing the rat in a supine position at regular intervals (˜20 seconds). The period of time required for righting from the supine to the prone position is used as an additional indicator of injury severity. Sham CCI control animals are surgically prepared and interfaced with the device but with no impact delivered to the brain. N=6-8 animals per group.

A single acute administration of abemaciclib after TBI.

Abemaciclib was dissolved in 0.9% saline, and then administered intravenously (i.v.) as a single acute dose (3 mg/kg, i.v.) at 0 hr, 3 hrs, or 6 hrs after TBI in rats. Sham and vehicle control animals received 1 ml saline (i.v.).

### Elevated Body Swing Test (EBST).

EBST was used to examine the lateral motor deficits 22˜24 hours following CCI. Briefly, each rat was placed into a Plexiglas box (50×50×20 cm), and allowed to habituate for 10 minutes and attain a neutral position as defined as having all four paws on the ground. Then the rat was held approximately 3 cm above the table and approximately 3 cm from the base of its tail. The left or rightward swings of each rat were recorded over 45 seconds. This was repeated 3 times with 2 minutes in between each test. Biased swinging behavior was calculated as follows: L/(L+R) (%) for left biased swings (L, left-biased swings; R, right-biased swings). The criterion for biased swinging behavior was set at 70% or higher. Statistical differences were determined using one-way ANOVA followed by Dunnett's post hoc test.

### Results

A single administration of abemaciclib (3 mg/kg, i.v., given 0 hr, 3 hrs, or 6 hrs post-TBI) improves motor function after TBI in rats.

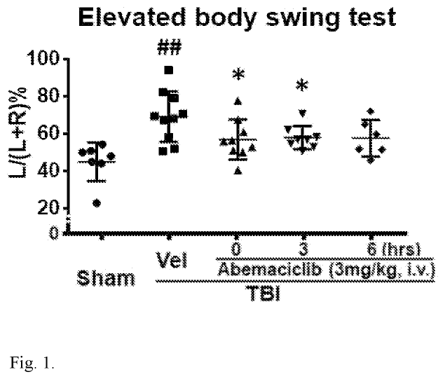

The data shows that TBI causes biased swinging to the contralateral side of TBI 24 hrs after TBI (#P<0.05 vs sham surgery, unpaired t-tests, FIG. 1). A single intravenous (i.v.) administration of abemaciclib (3 mg/kg, administered at 0 hr and 3 hrs post-TBI) significantly reduced the biased swinging behavior 24 hrs after TBI (*P<0.05 vs TBI/vehicle (Vel), unpaired t-tests, FIG. 1). Delayed abemaciclib administration (6 hrs post-TBI) reduced the motor deficits, though not significantly (FIG. 1 and unpublished data).

### Example 2

This example describes that a single treatment with a representative CDK4/CDK6 inhibitor increases hippocampal neuron survival in a representative experimental model of TBI.

CCI was used to produce TBI in adult male Sprague-Dawley rats as described in Example 1.

### Brain Sample Preparation and Immunohistochemistry.

Anesthetized rats were perfused with saline followed by 4% PFA at day 1 or day 16. Brain sections (50 μm) are cut at −20° C. and incubated in 0.3% H₂O₂ for 10 minutes to quench endogenous peroxidase, incubated with 3% horse blocking serum for 20 minutes, and then incubated overnight in primary antibody (mouse anti-NeuN, a mature neuron marker, 1:150, Millipore), then in biotinylated secondary antibody (Goat anti-mouse 1:1,000, Vector) for 0.5 h, then placed in Vectastain ABC reagent for 0.5 h and incubated in peroxidase substrate 3, 3′-diamino-benzidine (DAB) solution for 10 min.

### Stereological Cell Counts.

The total number of NeuN-positive neurons in CA2-3 of hippocampus were quantified bilaterally using stereological methods. The NeuN-positive cell counts were performed on a microscope (Nikon E600, Nikon) using commercial software (Stereo Investigator™8.0, Microbrightfield, Inc.). Serial sections cut at 50 μm thick were collected at Bregma-2.80 mm to Bregma-4.16 mm for a total of 10 sections per brain. Estimated numbers of the NeuN-positive neurons in the target brain region were generated by the Stereologer software using the following equation: ΣNobj=(EN)(1/SSF)(1/ASF) (1/TSF) (ref. 21). In this equation ΣNobj indicates the sum of objects sampled in the sections, SSF indicates the section sampling fraction, ASF indicates the area sampling fraction, and TSF indicates the thickness sampling fraction. Counting is performed by a blinded observer. Statistical differences are determined using ANOVA followed by Dunnett's post hoc test.

### Results

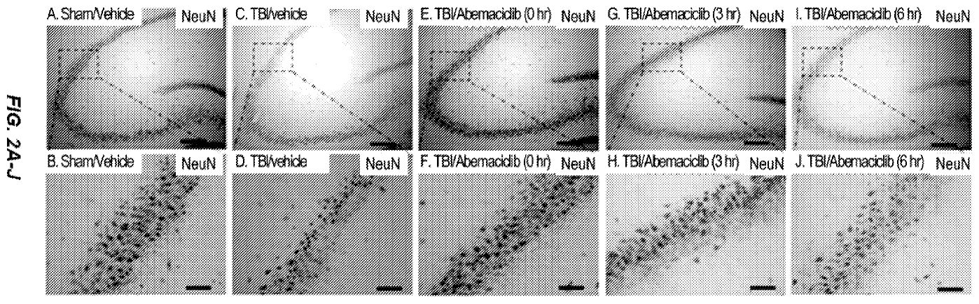

A single intravenous administration of abemaciclib (3 mg/kg, administered at 0 hr, 3 hrs, or 6 hrs post-TBI) promotes hippocampal neuron survival after TBI. FIGS. 2A-2J show fixed brain coronal sections stained with NeuN a described above.

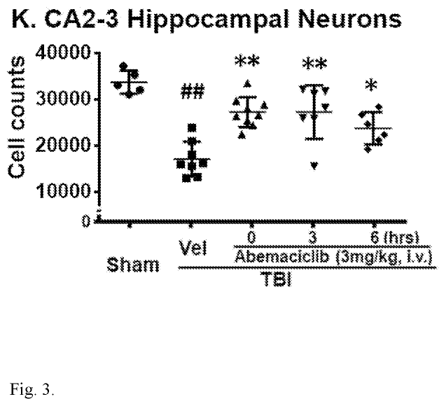

The NeuN⁺ cells in ipsilateral hippocampal CA2-3 were quantified using an unbiased Optical Dissector stereological method as described above (see FIG. 3). The data shows that TBI resulted in loss of hippocampal neurons in the ipsilateral side of TBI 24 hrs after TBI (##P<0.01 vs sham surgery controls, unpaired t-tests, FIG. 3). A single intravenous (i.v.) administration of abemaciclib (3 mg/kg, administered at 0 hr, 3 hrs, or 6 hrs post-TBI) significantly attenuated the loss of hippocampal neurons 24 hrs after TBI (**P<0.01 vs TBI/vehicle (Vel), unpaired t-tests, FIG. 3).

### REFERENCES

1 Faul, M., Xu, L., Wald, M. M. & Coronado, V. G. Traumatic brain injury in the United States: emergency department visits, hospitalizations, and deaths. Atlanta (GA): Centers for Disease Control and Prevention, National Center for Injury Prevention and Control. 7-23 (2010).
2 Dewan, M. C. et al. Estimating the global incidence of traumatic brain injury. Journal of neurosurgery, 1-18, doi: 10.3171/2017.10.JNS17352 (2018).
3 EnergiasMarketResearch. Traumatic Brain Injuries Treatment Market. https://www.energiasmarketresearch.com/global-traumatic-brain-injuries-treatment-market-size/(2018).
4 Liu, D. Z. & Ander, B. P. Cell cycle phase transitions: signposts for aberrant cell cycle reentry in dying mature neurons. Journal of Cytology & Histology 2, e101 (2011).
5 Liu, D. Z. & Ander, B. P. Cell cycle inhibition without disruption of neurogenesis is a strategy for treatment of aberrant cell cycle diseases: an update. ScientificWorldJournal 2012, 491737 (2012).
6 Liu, D. Z., Ander, B. P. & Sharp, F. R. Cell cycle inhibition without disruption of neurogenesis is a strategy for treatment of central nervous system diseases. Neurobiology of disease 37, 549-557 (2010).
7 Summy, J. M. & Gallick, G. E. Src family kinases in tumor progression and metastasis. Cancer Metastasis Rev 22, 337-358 (2003).
8 Matsuoka, T. & Yashiro, M. Rho/ROCK signaling in motility and metastasis of gastric cancer. World J Gastroenterol 20, 13756-13766, doi: 10.3748/wjg.v20.i38.13756 (2014).
9 Shi, Y. et al. Rapid endothelial cytoskeletal reorganization enables early blood-brain barrier disruption and long-term ischaemic reperfusion brain injury. Nat Commun 7, 10523, doi: 10.1038/ncomms10523 (2016).
10 Liu, D. Z. et al. Blood-brain barrier breakdown and repair by Src after thrombin-induced injury. Annals of neurology 67, 526-533, doi: 10.1002/ana.21924 (2010).
11 Putzke, A. P. et al. Metastatic progression of prostate cancer and e-cadherin regulation by zeb1 and SRC family kinases. Am J Pathol 179, 400-410, doi: 10.1016/j.ajpath.2011.03.028 (2011).
12 Wu, Z. et al. Autophagy Blockade Sensitizes Prostate Cancer Cells towards Src Family Kinase Inhibitors. Genes Cancer 1, 40-49, doi: 10.1177/1947601909358324 (2010).
13 Nam, J. S., Ino, Y., Sakamoto, M. & Hirohashi, S. Src family kinase inhibitor PP2 restores the E-cadherin/catenin cell adhesion system in human cancer cells and reduces cancer metastasis. Clin Cancer Res 8, 2430-2436 (2002).
14 Liu, D. et al. Inhibition of Src Family Kinases Protects Hippocampal Neurons and Improves Cognitive Function after Traumatic Brain Injury. Journal of neurotrauma 31, 1268-1276, doi: 10.1089/neu.2013.3250 (2014).
15 Liu, D. Z. et al. Src kinase inhibition decreases thrombin-induced injury and cell cycle re-entry in striatal neurons. Neurobiology of disease 30, 201-211 (2008).
16 Abemaciclib for breast cancer. Aust Prescr 43, 94-95, doi: 10.18773/austprescr.2020.026 (2020).
17 Braal, C. L. et al. Inhibiting CDK4/6 in Breast Cancer with Palbociclib, Ribociclib, and Abemaciclib: Similarities and Differences. Drugs 81, 317-331, doi: 10.1007/s40265-020-01461-2 (2021).
18 Tolaney, S. M. et al. A Phase II Study of Abemaciclib in Patients with Brain Metastases Secondary to Hormone Receptor-Positive Breast Cancer. Clin Cancer Res 26, 5310-5319, doi: 10.1158/1078-0432.CCR-20-1764 (2020).
19 Nair, A. B. & Jacob, S. A simple practice guide for dose conversion between animals and human. J Basic Clin Pharm 7, 27-31, doi: 10.4103/0976-0105.177703 (2016).
20 Nosengo, N. Can you teach old drugs new tricks? Nature 534, 314-316, doi: 10.1038/534314a (2016).
21 Lee, L. L., Galo, E., Lyeth, B. G., Muizelaar, J. P. & Berman, R. F. Neuroprotection in the rat lateral fluid percussion model of traumatic brain injury by SNX-185, an N-type voltage-gated calcium channel blocker. Experimental neurology 190, 70-78, doi: S0014-4886 (04) 00275-4 [pii] 10.1016/j.expneurol.2004.07.003 (2004).

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described. All publications, patents, and patent applications cited in this specification are herein incorporated by reference as if each individual publication, patent, or patent application were specifically and individually indicated to be incorporated by reference.

### EXEMPLARY EMBODIMENTS

Exemplary embodiments provided in accordance with the presently disclosed subject matter include, but are not limited to, the claims and the following embodiments:
1. A method for treating traumatic brain injury (TBI) in a human subject in need thereof, comprising administering a composition comprising a therapeutically effective amount of a selective cyclin-dependent kinase 4 (CDK4)/cyclin-dependent kinase 6 (CDK6) inhibitor to the subject, thereby treating TBI in the human subject.
2. The method of embodiment 1, wherein the selective CDK4/CDK6 inhibitor is selected from the group consisting of abemaciclib (LY2835219), palbociclib (PD-0332991), ribociclib (LEE011), trilaciclib (CAS No. 1374743-00-6), a salt thereof, and a combination thereof.
3. The method of embodiment 1 or 2, wherein the subject is an adolescent or adult human.
4 The method of any one of embodiments 1 to 3, wherein the therapeutically effective amount comprises a dose of the selective CDK4/CDK6 inhibitor of about 20 mg/day to about 100 mg/day.
5. The method of any one of embodiments 1 to 4, wherein the selective CDK4/CDK6 inhibitor is administered in one or more doses.
6. The method of any one of embodiments 1 to 5, wherein the inhibitor is administered in a single dose.
7. The method of embodiment 6, wherein the inhibitor is administered during the acute stage of TBI.
8. The method of embodiment 7, wherein the inhibitor is administered within about 0.1 to about 9 hours following TBI.
9. The method of any one of embodiments 6 to 8, wherein the single dose comprises about 20 mg to about 100 mg of the inhibitor.
10. The method of any one of embodiments 6 to 9, wherein the single dose comprises about 30 mg of the inhibitor.
11. The method of any one of embodiments 1 to 10, wherein the TBI is classified as mild, moderate or severe TBI.
12. The method of embodiment 11, wherein the TBI is classified as mild TBI, and the composition is administered to the subject orally.
13. The method of embodiment 11, wherein the TBI is classified as moderate to severe TBI, and the composition is administered to the subject intravenously.
14. The method of any one of embodiments 1 to 13, wherein the composition has decreased side effects compared to a dose of the inhibitor administered at 100 mg or greater one time per day.
15. The method of embodiment 14, wherein the side effects are selected from those in a row of Table 2.
16. The method of any one of embodiments 1 to 15, wherein the composition increases motor function in the subject following TBI as determined by Assessment of Motor and Process Skills (AMPS).
17. The method of any one of embodiments 1 to 16, wherein the composition promotes hippocampal neuronal survival following TBI.
18. The method of any one of embodiments 1 to 17, wherein the composition improves cognitive function in the subject as measured by the Montreal Cognitive Assessment, the Overall Test Battery Mean, a latent ability composite score, the Neuropsychological Deficit Score, or a combination thereof.
19. The method of any one of embodiments 1 to 18, wherein administering the composition to the subject results in decreased symptoms of TBI as determined by the Glasgow outcome scale extended (GOSE).
20. The method of embodiment 19, wherein administering the composition to the subject results in a 2 level improvement on the GOSE at 6 months after the TBI occurred.
21. The method of any one of embodiments 1 to 20, wherein the composition results in decreased symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof.
22. The method of any one of embodiments 1 to 21, wherein the composition results in decreased symptoms of TBI-induced brain damage selected from the group consisting of intracranial hemorrhage, brain hematoma, motor deficits, disruption of the blood brain barrier, brain edema, and a combination thereof.
23. The method of any one of embodiments 1 to 22, wherein the composition results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof.
24. The method of any one of embodiments 1 to 23, wherein the composition further comprises a pharmaceutically acceptable carrier or excipient.
25. The method of any one of embodiments 1 to 24, wherein the composition is administered to the subject intravenously or orally.

What is claimed is:

1. A method for treating traumatic brain injury (TBI) in a human subject in need thereof, comprising administering a composition comprising a therapeutically effective amount of a selective cyclin-dependent kinase 4 (CDK4)/cyclin-dependent kinase 6 (CDK6) inhibitor to the subject, thereby treating TBI in the human subject.

2. The method of claim 1, wherein the selective CDK4/CDK6 inhibitor is selected from the group consisting of abemaciclib (LY2835219), palbociclib (PD-0332991), ribociclib (LEE011), trilaciclib (CAS No. 1374743-00-6), a salt thereof, and a combination thereof.

3. The method of claim 1, wherein the subject is an adolescent or adult human.

4. The method of claim 1, wherein the therapeutically effective amount of the selective CDK4/CDK6 inhibitor comprises a dose of about 20 mg/day to about 100 mg/day.

5. The method of claim 1, wherein the selective CDK4/CDK6 inhibitor is administered in one or more doses.

6. The method of claim 5, wherein the inhibitor is administered in a single dose.

7. The method of claim 6, wherein the inhibitor is administered during the acute stage of TBI.

8. The method of claim 7, wherein the inhibitor is administered within about 0.1 to about 9 hours following TBI.

9. The method of claim 6, wherein the single dose comprises about 20 mg to about 100 mg of the inhibitor.

10. The method of claim 9, wherein the single dose comprises about 30 mg of the inhibitor.

11. The method of claim 1, wherein the TBI is classified as mild, moderate or severe TBI.

12. The method of claim 11, wherein the TBI is classified as mild TBI, and the composition is administered to the subject orally.

13. The method of claim 11, wherein the TBI is classified as moderate to severe TBI, and the composition is administered to the subject intravenously.

14. The method of claim 1, wherein the composition has decreased side effects compared to a dose of the inhibitor administered at 100 mg or greater one time per day.

15. The method of claim 14, wherein the side effects are selected from those in a row of Table 2.

16. The method of claim 1, wherein the composition increases motor function in the subject following TBI as determined by Assessment of Motor and Process Skills (AMPS).

17. The method of claim 1, wherein the composition promotes hippocampal neuronal survival following TBI.

18. The method of claim 1, wherein the composition improves cognitive function in the subject as measured by the Montreal Cognitive Assessment, the Overall Test Battery Mean, a latent ability composite score, the Neuropsychological Deficit Score, or a combination thereof.

19. The method of claim 1, wherein administering the composition to the subject results in decreased symptoms of TBI as determined by the Glasgow outcome scale extended (GOSE).

20. The method of claim 19, wherein administering the composition to the subject results in a 2 level improvement on the GOSE at 6 months after the TBI occurred.

21. The method of claim 1, wherein the composition results in decreased symptoms of TBI selected from the group consisting of headache, nausea or vomiting, fatigue or drowsiness, speech problems, dizziness or loss of balance, blurred vision, ringing in the ears, a bad taste in the mouth, changes in the ability to smell, sensitivity to light or sound, loss of consciousness, confusion, disorientation, memory or concentration problems, mood changes or mood swings, depression, anxiety, difficulty sleeping, sleeping more than usual, convulsions or seizures, dilation of one or both pupils of the eyes, clear fluids draining from the nose or ears, inability to awaken from sleep, weakness or numbness in fingers and toes, loss of coordination, agitation, combativeness, slurred speech, coma, and a combination thereof.

22. The method of claim 1, wherein the composition results in decreased symptoms of TBI-induced brain damage selected from the group consisting of intracranial hemorrhage, brain hematoma, motor deficits, disruption of the blood brain barrier, brain edema, and a combination thereof.

23. The method of claim 1, wherein the composition results in decreased symptoms of primary or secondary TBI selected from the group consisting of accumulation of intracellular calcium in neurons, cell depolarization, excitotoxic release of glutamate, disruption of ionic gradients, impaired mitochondrial function, elevated reactive oxygen species, neuroinflammation, and a combination thereof.

24. The method of claim 1, wherein the composition further comprises a pharmaceutically acceptable carrier or excipient.

25. The method of claim 1, wherein the composition is administered to the subject intravenously or orally.
